(19) United States
(12) Patent Application Publication
(10) Pub. No.: US 2020/0329719 A1
(43) Pub. Date: Oct. 22, 2020

(54) SYSTEM AND METHOD FOR REMOTE MANAGEMENT OF SMART GRINDING DEVICE

(71) Applicant: GENERATION INFINITE MACHINES PVT LTD, Chennai (IN)

(72) Inventor: Muthukumaran Subbiah, Tirunelveli (IN)

(73) Assignee: GENERATION INFINITE MACHINES PVT LTD, Chennai (IN)

(21) Appl. No.: 16/959,233

(22) PCT Filed: Dec. 26, 2018
(86) PCT No.: PCT/IN2018/050884

§ 371 Date: Jun. 30, 2020

(30) Foreign Application Priority Data
Jan. 1, 2018 (IN) 201841000043

(51) Int. Cl. A21C 13/00; B02C 25/00; G01N 33/02; A47J 44/02; A47J 43/044; A47J 43/07; A21C 1/02; A21C 1/12; A21C 1/14; A21C 1/00

(52) U.S. Cl. CPC A21C 13/00; A47J 2203/00; G01N 33/02; A47J 44/02; A47J 43/044; A47J 43/0711; A21C 1/02; A21C 1/12; A21C 1/146; A21C 1/1455; A21C 1/003; A21C 1/006; A47J 2202/00; A47J 2043/04454; A47J 2043/0733; B02C 25/00

(57) ABSTRACT

A system for remote management of smart grinding device is provided. The system includes a device configured for performing a plurality of functions based on a one or more inputs received. The plurality of functions includes identifying, weighing, dispensing, cleaning, soaking, grinding, fermenting, altering the proportions and storing of one or more ingredients for preparation of an edible composition. The system also includes a communication interface configured for communicating the one or more inputs to the device and further communicating a real time information related to the plurality of functions performed, to a remote server via a wireless network. The system also includes the remote server configured for continuously analyzing the real time information related to the plurality of functions performed by the device by using artificial intelligence (AI) for generating the one or more inputs for the plurality of functions.

### RELATED APPLICATIONS

This application claims priority from Indian Provisional patent application numbered 201841000043 titled “System and method for remote management of smart grinding device” filed on 1 Jan. 2018, as claimed in PCT application No. PCT/IN2018/050884 filed on 26 Dec. 2018 from which application is derived.

### FIELD OF TECHNOLOGY

This disclosure relates generally to remote management of a smart grinding and blending device and more particularly to a device configured for performing a plurality of functions like identifying, weighing, dispensing, cleaning, soaking, grinding, fermenting, altering the proportions and storing of one or more ingredients for preparation of an edible composition based on a one or more inputs received from a remote server.

### BACKGROUND

Wet grinding has been one of the most tedious processes involved in food preparation. It takes enormous time and effort to measure, wash, soak and prepare the ingredients preceding the actual grinding process. Furthermore, the grinding process requires continuous monitoring for the consistency of the batter and water requirements. In addition, fermentation of the batter is an important step in making some food preparations like Idly, Dosa, Vada and the like. Moreover, fermentation is a temperature-dependent process and as a result it is impossible to achieve a similar taste in different parts of the world, especially in cold regions. Particularly, a device equipped with a fermentation and a refrigeration unit is needed to maintain the optimum environmental conditions required for the storage of the end product till further use.

Conventional wet grinders used for making batter utilize heavy stones and complex machinery that cannot be easily cleaned by the consumers. The existing tabletop and tilting wet grinders have reduced the pain, but still fail to eliminate it. Therefore, a need for auto cleaning feature in the wet grinders to wash and clean the entire appliance without any manual labour is required.

Time management is very essential in day-to-day life. Several existing grinding devices are made to ease laborious manual work. The plethora of wet grinders currently available in the market do not eliminate the need of manual supervision and/or intervention. As a result, a device is required that grinds the ingredients without any manual intervention by pumping in the required quantity of water at proper intervals.

To overcome at least some of the above mentioned problems, there exists a need for an automatic wet grinding device, which manifests a compact kitchen apparatus that automatically weighs, soaks, grinds the ingredients and stores the slurry in optimum temperature till used for cooking. A grinding device is needed, which can be remotely operated using handheld devices like smart phones, tablets, etc. using Internet based connectivity, like Wi-Fi, GSM, etc.

### SUMMARY

The following summary is illustrative only and is not intended to be in any way limiting. In addition to the illustrative aspects, example embodiments, and features described above, further aspects, example embodiments of the present disclosure will become apparent by reference to the drawings and the following detailed description.

Embodiments of the present disclosure a system. The system includes a device configured for performing a plurality of functions based on a one or more inputs received. The plurality of functions includes identifying, weighing, dispensing, cleaning, soaking, grinding, fermenting, altering the proportions and storing of one or more ingredients for preparation of an edible composition. The system also includes a communication interface configured for communicating the one or more inputs to the device and further communicating a real time information related to the plurality of functions performed, to a remote server via a wireless network. The system also includes the remote server configured for continuously analysing the real time information related to the plurality of functions performed by the device, by using artificial intelligence (AI) for generating the one or more inputs for the plurality of functions.

Also disclosed is a method for performing a plurality of functions, by a device based on a one or more inputs received. The method includes communicating the one or more inputs to a device, for performing a plurality of functions for producing a final grinded mixture of ingredients for preparation of an edible composition. Furthermore, the method includes implementing the operation of the plurality of functions, by the device. The plurality of functions includes identifying, weighing, dispensing, cleaning, soaking, grinding, altering the proportions, fermenting and storing of a one or more ingredients for preparation of an edible composition. The method further includes communicating a real time information related to the plurality of functions performed, to a remote server via a wireless network. In addition, the method includes analyzing the real time information by the remote server, related to the plurality of functions performed by the device, by using artificial intelligence (AI) for generating the one or more inputs for the plurality of functions and for making alteration to maintain the taste and quality of the edible composition.

The summary above is illustrative only and is not intended to be in any way limiting. Further aspects, exemplary embodiments, and features will become apparent by reference to the drawings and the following detailed description.

### BRIEF DESCRIPTION OF THE FIGURES

These and other features, aspects, and advantages of example embodiments will be better understood when the following detailed description is read with reference to the accompanying drawings in which like characters represent like parts throughout the drawings, wherein:

FIG. 1 is a block diagram of a system illustrating remote management of smart grinding and blending device configured for performing a plurality of functions like identifying, weighing, dispensing, cleaning, soaking, grinding, fermenting, altering the proportions and storing of one or more ingredients for preparation of an edible composition based on a one or more inputs received from a remote server, according to an example embodiment of the present disclosure;

FIG. 2 is a flow chart illustrating a method for performing a plurality of functions, by a device based on a one or more inputs received from a remote server, according to an embodiment of the present disclosure;

FIG. 3 illustrates a storage unit of the device of system of FIG. 1, according to an embodiment of the present disclosure;

FIG. 4 illustrates a connecter chamber unit of the device of system of FIG. 1, according to an embodiment of the present disclosure;

FIG. 5 illustrates an electronic module of the device of system of FIG. 1, according to an embodiment of the present disclosure;

FIG. 6 illustrates a motor module of the device of system of FIG. 1, according to an embodiment of the present disclosure;

FIG. 7 illustrates a mounting unit of the device of system of FIG. 1, according to an embodiment of the present disclosure;

FIG. 8 illustrates a grinding module of the device of system of FIG. 1, according to an embodiment of the present disclosure;

FIG. 9 illustrates a water and fermentation module of the device of system of FIG. 1, according to an embodiment of the present disclosure;

FIG. 10 illustrates a base and body module of the device of system of FIG. 1, according to an embodiment of the present disclosure;

FIG. 11 illustrates a cross sectional view of a hopper unit of a hopper module of the device of system of FIG. 1, according to an embodiment of the present disclosure;

FIG. 12 illustrates a cross sectional view of a valve unit of the hopper module of the device of system of FIG. 1, according to an embodiment of the present disclosure;

FIG. 13 illustrates a cross sectional view of a connecting chamber module of the device of system of FIG. 1, according to an embodiment of the present disclosure;

FIG. 14 illustrates a cross sectional view of an electronic module of the device of system of FIG. 1, according to an embodiment of the present disclosure;

FIG. 15 illustrates a cross sectional view of a motor module of the device of system of FIG. 1, according to an embodiment of the present disclosure;

FIG. 16 illustrates a cross sectional view of a base mount module of the device of system of FIG. 1, according to an embodiment of the present disclosure;

FIG. 17 illustrates a cross sectional view of a blade unit of a grinding module of the device of system of FIG. 1, according to an embodiment of the present disclosure;

FIG. 18 illustrates a cross sectional view of a grinding unit of a grinding module of the device of system of FIG. 1, according to an embodiment of the present disclosure;

FIG. 19 illustrates a cross sectional view of a water and a fermentation module of the device of system of FIG. 1, according to an embodiment of the present disclosure;

FIG. 20 illustrates a cross sectional view of a base module of the device of system of FIG. 1, according to an embodiment of the present disclosure;

FIG. 21 illustrates a cross sectional view of an overall assembly of the device of system of FIG. 1, implemented according an embodiment of the present disclosure, according to an embodiment of the present disclosure;

Further, skilled artisans will appreciate that elements in the figures are illustrated for simplicity and may not have necessarily been drawn to scale. Furthermore, in terms of the construction of a device, one or more components of the device may have been represented in the figures by conventional symbols, and the figures may show only those specific details that are pertinent to understanding the embodiments of the present disclosure so as not to obscure the figures with details that will be readily apparent to those of ordinary skill in the art having the benefit of the description herein.

### DETAILED DESCRIPTION

In addition to the illustrative aspects, exemplary embodiments, and features described above, further aspects, exemplary embodiments of the present disclosure will become apparent by reference to the drawings and the following detailed description.

For the purpose of promoting an understanding of the principles of the invention, reference will now be made to the embodiments illustrated in the figures and specific language will be used to describe the same. It will nevertheless be understood that no limitation of the scope of the invention is thereby intended, such alterations and further modifications in the illustrated system, and such further applications of the principles of the invention as illustrated therein being contemplated as would normally occur to one skilled in the art to which the invention relates.

It will be understood by those skilled in the art that the foregoing general description and the following detailed description are exemplary and explanatory of the invention and are not intended to be restrictive thereof.

The terms “comprises”, “comprising”, or any other variations thereof, are intended to cover a non-exclusive inclusion, such that a process or method that comprises a list of steps does not comprise only those steps but may comprise other steps not expressly listed or inherent to such process or method. Similarly, one or more devices or sub-systems or elements or structures or components proceeded by “comprises . . . a” does not, without more constraints, preclude the existence of other devices or other sub-systems or other elements or other structures or other components or additional devices or additional sub-systems or additional elements or additional structures or additional components. Appearances of the phrase “in an embodiment”, “in another embodiment” and similar language throughout this specification may, but do not necessarily, all refer to the same embodiment.

At least one exemplary embodiment is generally directed towards methods and systems for performing a plurality of functions based on a one or more inputs received. The plurality of functions includes identifying, weighing, dispensing, cleaning, soaking, grinding, fermenting, altering the proportions and storing of one or more ingredients for preparation of an edible composition. The system also includes a communication interface configured for communicating the one or more inputs to the device and further communicating a real time information related to the plurality of functions performed, to a remote server via a wireless network. The system also includes the remote server configured for continuously analyzing the real time information related to the plurality of functions performed by the device by using an artificial intelligence (AI) for generating the one or more inputs for the plurality of functions.

Unless otherwise defined, all technical and scientific terms used herein have the same meaning as commonly understood by one of ordinary skill in the art to which this invention belongs. The system, methods, and examples provided herein are illustrative only and not intended to be limiting.

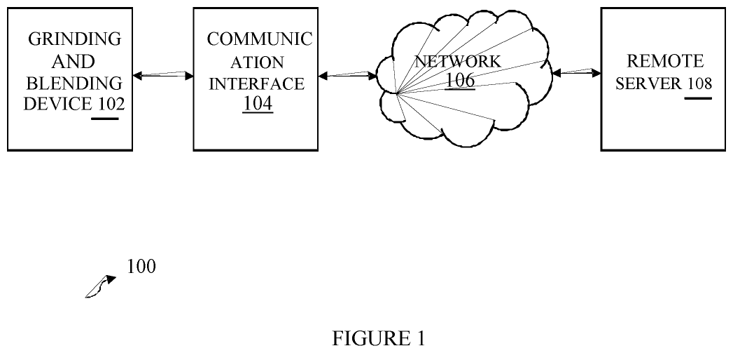

FIG. 1 is a block diagram of a system 100 illustrating a remote management of smart grinding and blending device 102 configured for performing a plurality of functions like identifying, weighing, dispensing, cleaning, soaking, grinding, fermenting, altering the proportions and storing of one or more ingredients for preparation of an edible composition based on a one or more inputs received from a remote server 108, according to an example embodiment of the present disclosure. In particular, the system 100 includes a device 102, a communication interface 104, a network 106 and a remote server 108. FIG. 1 is explained with respect to a single device 102 for example the smart grinding and blending device 102. However, it should be noted that a plurality of devices 102 can also be similarly included in the system 100.

Referring to FIG. 1, the system 100 illustrates a device 102 configured for performing a plurality of functions based on a one or more inputs received. In one embodiment, the plurality of functions are identifying, weighing, dispensing, cleaning, soaking, grinding, fermenting, altering the proportions and storing of one or more ingredients for preparation of an edible composition.

In one example embodiment, the device 102 is a smart grinding and blending device communicatively coupled to the remote server 108 further enabling a user to send one or more inputs from a hand-held device via an interface. In another example embodiment, the device 102 is an IoT (internet of things) enabled device configured for remote operation using handheld devices.

In one embodiment, the device 102 includes a plurality of units. Furthermore, the plurality of units includes a plurality of sensors configured for collecting and communicating real time information related to the plurality of functions performed by the device, to the remote server 108 via the communication interface 104 for the artificial intelligence (AI) to analyze and communicate inputs. The system 100 also includes a communication interface 104 configured for communicating the one or more inputs to the device 102 and further communicating a real time information related to the plurality of functions performed, to a remote server 108 via a wireless network 104. The remote server 108 is configured for continuously analyzing the real time information related to the plurality of functions performed by the device 102 by using artificial intelligence (AI) for generating the one or more inputs for the plurality of functions. The artificial intelligence (AI) as described herein refers to one or more modules operating at the remote server 108 configured for running one or more machine learning models based on training data to learn and predict the plurality of functions of the system 100. For instance, based on a geographical coordinates data of ‘n’ samples of batter preparation, the artificial intelligence module predicts the optimum temperature range for a queried geographical location and recommends the optimum values to the user.

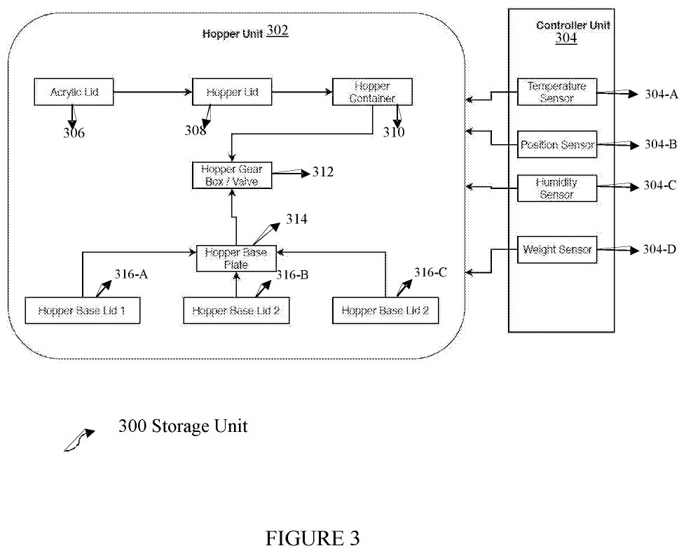

As described above, the device 102 includes a plurality of units. In one embodiment, one of the unit of the plurality of unit is a storage unit. The storage unit (as shown in FIG. 3) includes one or more hoppers configured for storing the one or more ingredients separately and free from contamination. Furthermore, the storage unit includes the plurality of sensors configured for identifying the quality of one or more ingredients, weighing the one or more ingredients stored in the hopper, monitoring the temperature in the hopper, checking a position of a hopper valve between opening and closing, monitoring humidity and water vapor content in the hopper and further communicating the real time sensed information to the remote server 108 via the communication interface 104 for the artificial intelligence (AI) to perform alteration for maintaining at least the taste, texture, shelf life and quality of the edible composition.

The storage unit facilitates the storage of different types of ingredients separately in each of the compartment. The storage unit is configured with a door to prevent the contamination of the ingredients stored and to maintain a required temperature within the storage area. In one example, the storage unit may include infra-red/optic sensors to recognize the variety or quality of ingredients. The sensors are configured to collect this information and transmit to the remote server 108 (cloud) where the artificial intelligence (AI) re-alters the recipe accordingly. The bottom of the storage unit are equipped with weight sensors and/or level indicators to measure the exact quantity available in the storage containers and transmit the information to the user as an alert via remote server 108 (cloud). The weight sensors ensure that the ingredients are measured exactly as per user's requirement.

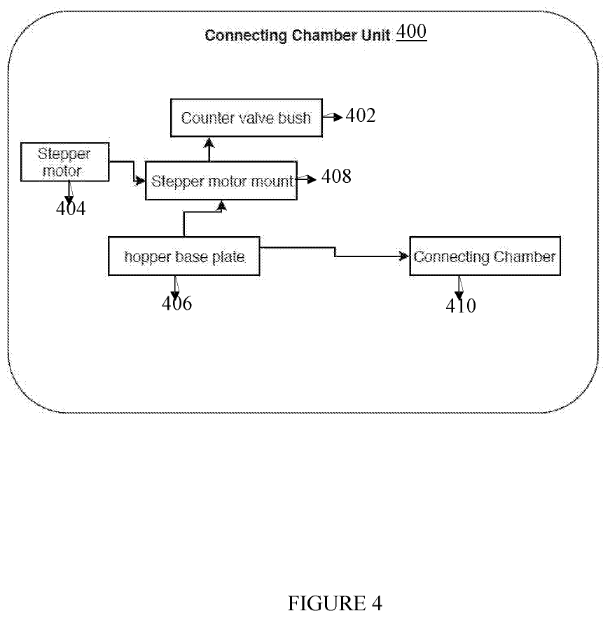

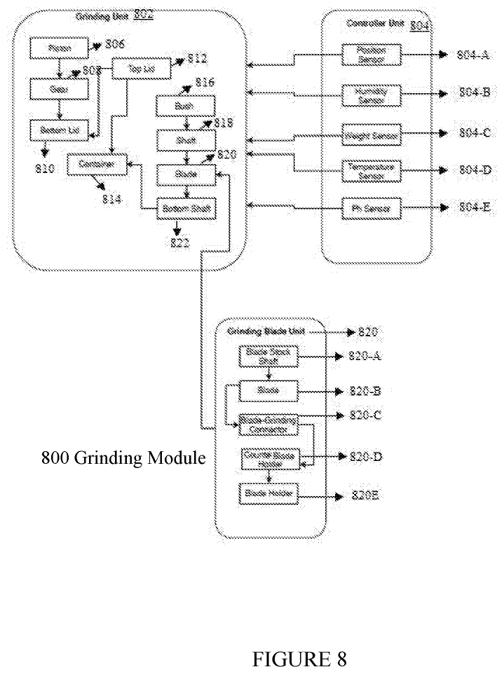

Furthermore, the bottom of the storage unit is equipped with valves that are activated via motors and controlled electronically to regulate the flow of ingredients to the connecting/soaking unit. The connecting unit (as shown in FIG. 4) is configured for a passage of the one or more ingredients to a grinding unit (as shown in FIG. 8). In one example, the connecting unit includes encoders configured to translate rotary or linear motion into a digital signal.

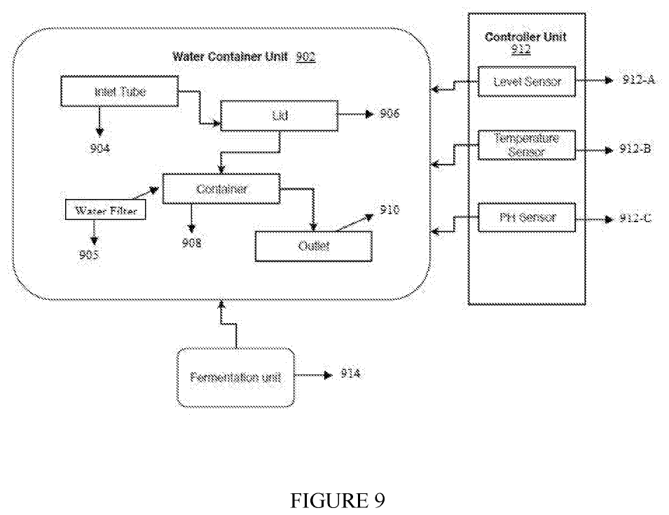

In another embodiment, one of the unit of the plurality of unit of the device 102 is the soaking unit configured for soaking the one or more ingredients using fresh water from a water container comprising water level sensors. In another embodiment, one of the unit of the plurality of unit of the device 102 is the fermentation unit (as shown in FIG. 9) configured to produce hot warm air for fermenting the final grinded mixture of the ingredients. In another embodiment, one of the unit of the plurality of unit of the device 102 is the refrigeration unit configured to produce cool air for maintaining a refrigeration effect for the final grinded mixture of the ingredients.

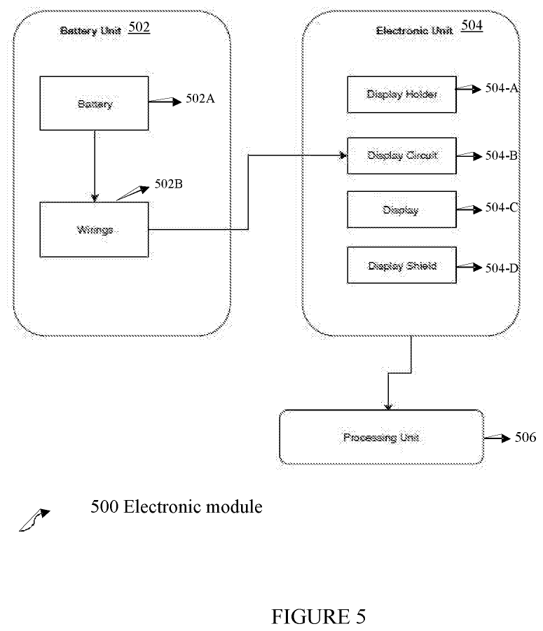

In another embodiment, one of the unit of the plurality of unit of the device 102 is the controller unit. The controller unit includes a temperature sensor configured for monitoring the temperature in a grinding container and a water container. The controller unit includes a PH sensor configured for monitoring the Ph level of the water for acid and base value and for further monitoring the acid and base value of the final grinded mixture of ingredients. The controller unit includes a microbial sensor configured for monitoring the growth of unwanted microbes and prevent contamination of the water and the final grinded mixture of ingredients. The controller unit includes a gas sensor, a pressure sensor and a taste bud sensor configured for collecting physiochemical parameters of the final grinded mixture of ingredients. In another embodiment, one of the unit of the plurality of unit of the device 102 is an electronic unit (as shown in FIG. 5). The electronic unit includes a battery for sustaining the operation of the device during power failure; a power regulation unit for monitoring any fluctuation in power using current and voltage sensors; a display and a processing unit.

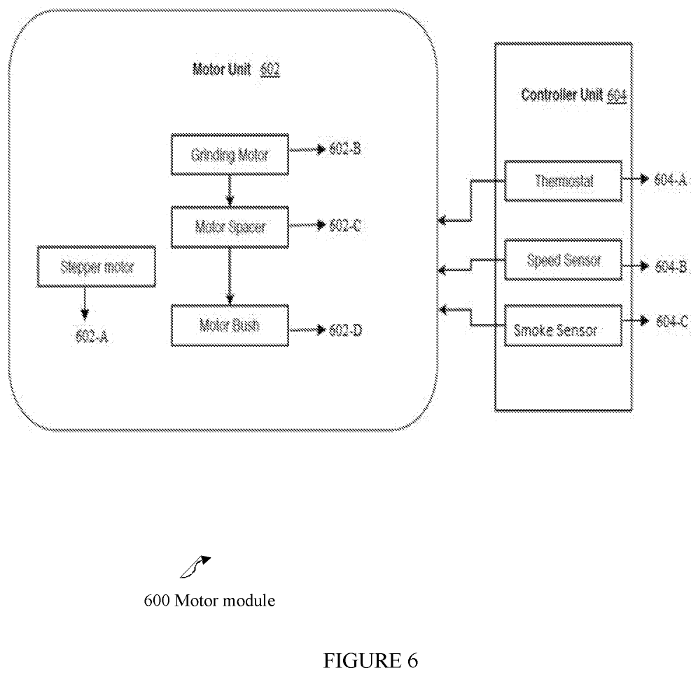

In another embodiment, one of the unit of the plurality of unit of the device 102 is a motor unit (as shown in FIG. 6). The motor unit includes a grinding motor configured for grinding the one or more ingredients to produce a final grinded mixture of ingredients for preparation of an edible composition. The motor unit includes the plurality of sensors configured for monitoring the temperature produced by the grinding motor and measuring the speed (RPM) of a motor shaft of the grinding motor and detecting a smoke. In yet another embodiment, the device 102 further includes a sensor management module (not shown) of the circuit for monitoring the quality of plurality of sensors and a plurality of lock sensors configured for monitoring the precise position of the several units and its parts.

In some embodiments, the system 100 disclosed herein may be explained by considering an example scenario for grinding batter required for making Idly, Dosa, Vada, Appam, Paniyaram, Crepes, Pancakes and other foods that involve wet grinding like Chutneys, Hummus and the like.

In one example embodiment, the system 100 illustrates an automatic wet grinding device 102, which manifests a compact kitchen apparatus that automatically weighs, soaks, grinds the one or more ingredients (for example rice and pulses) and stores the slurry at an optimum temperature till used for cooking. The wet grinding device 102 can be remotely operated using handheld devices. For conciseness, the device 102 is a compact, automatic, smart wet grinding device. In one example, the device 102 may be used as an end-to-end solution of food batter making process. This device 102 can be used to schedule and grind batter required for making Idly, Dosa, Vada, Appam, Paniyaram, Crepes, Pancakes and other foods that involve wet grinding like Chutneys, Hummus, etc. The device 102 may receive user's input via the capacitive touch screen display or remotely from any hand held device connected to the network 106. On receiving the input, the device 102 may measure, dispense, wash, soak and grind the required amount of ingredients automatically according to the schedule.

The soaking chamber receives the ingredients from the ingredient storage container via respective inlets. The water is pumped in via the water inlet containing water flow sensors to ensure the rate of flow and air is blowed through an air blower to wash the ingredients. The water outlet disposes the waste water out. The ingredients are soaked again using fresh water from the water storage unit that contains a water container equipped with water level sensors. The water input can be given manually or connected to a water purifying system. The water level indicators ensure the required amount of water to be collected, stored, used and send alerts to the user, if otherwise, remote server 108 (cloud).

The grinding unit receives the ingredients to be grinded via the connecting outlet and the valve seals the container to ensure there is no leakage. The grinding unit may equip either and/or a set of blades or graphite stones in order to crush and grind the raw/soaked ingredients that are powered by AC/DC motor controlled by an IoT circuit. The grinding time and speed are continuously monitored through an RPM sensor and the input is analysed by artificial intelligence (AI) in the remote server 108. A temperature sensor is affixed to the motor that acts as a cut-off switch in case of abnormal heating of the motor. The quality of the motor is also analysed using the information collected by a quality sensor. The motor also cuts off at regular intervals to ensure no extra heat is generated. The grinding unit receives water at even intervals for maintaining ease of grinding and maintaining consistency of the batter. The chamber also equips other sensors like CO2 sensor, pH sensor and taste bud sensor, which help in collecting physiochemical parameters of batter and sends it to the cloud for the artificial intelligence (AI) in the remote server 108 to analyze and make necessary recipe changes to ensure there is no change in the taste and quality of the end product.

The batter storage unit performs two tasks of fermentation and refrigeration of the batter under appropriate conditions. The fermentation unit senses the environmental conditions through thermostats and maintains the temperature inside the chamber irrespective of the geographical location. The fermentation unit is equipped with a coil that heats the air if required, which is circulated inside the chamber to maintain the temperature at about 28-38 degree Celsius. The refrigeration unit stores the batter in refrigerated conditions at about 4-8 degree Celsius after optimum fermentation is received that is sensed and analysed using pH and taste bud sensors.

The device 102 further includes a plurality of lock sensors in order to sense if all the different parts of the device are secured in right places. The device 102 is built in such a way that it can be called modular. The modular built allows the device 102 to be dismantled and the particular part to be replaced in case of maintenance and repair. The modular parts are controlled by a digital circuit which can also be ejected, if needed. The device 102 houses a smoke sensor to detect in case of any fire crisis in the machine.

The device 102 includes a communication interface 104 (for example, both a Wi-Fi and GSM module) to allow access remotely using any handheld device through a smartphone application designed for both Android and iOS devices. The device 102 can also be enabled using Voice assistants like Alexa, Google Assistant, Siri etc.

Furthermore, the remote server 108 and inbuilt AI system collects information from a plurality of individual devices (102) in every geographical location and continuously analyses using machine learning algorithms. These analytics help achieve similar results in every consumer household and satisfy the consumer needs by giving inputs to the user in terms of recipes, variety and quality of the ingredient used.

In some other embodiments, a manner in which the production of a final grinded mixture of ingredients is implemented for preparation of an edible composition is described in further detail below.

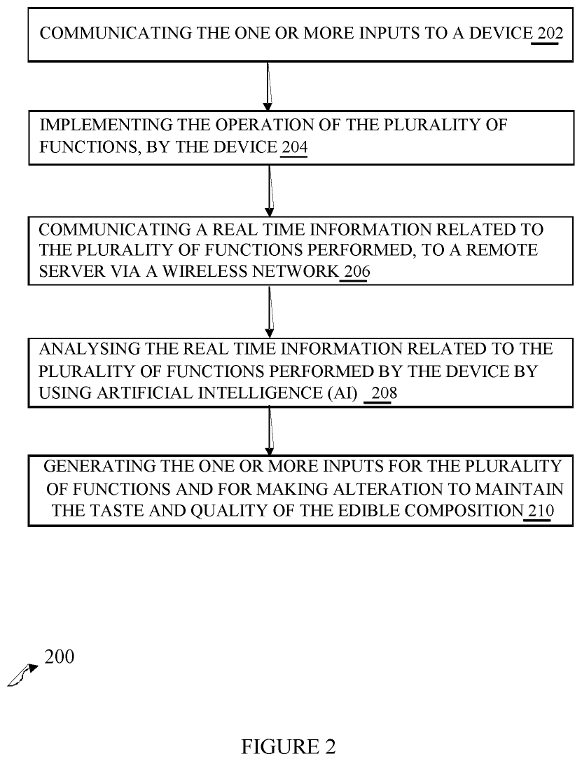

FIG. 2 is a flow chart illustrating a method for performing a plurality of functions, by a device based on a one or more inputs received from a remote server, according to an embodiment of the present disclosure. FIG. 2 may be described from the perspective of a controller that is configured to execute computer-readable instructions to carry out the functionalities of the above described modules of the system 100 shown in FIG. 2. In particular, the steps as described in FIG. 2 may be executed for performing a plurality of functions based on a one or more inputs received; wherein the plurality of functions includes identifying, weighing, dispensing, cleaning, soaking, grinding, fermenting, altering the proportions and storing of a one or more ingredients for preparation of an edible composition.

At step 202, one or more inputs are communicated to a device for performing a plurality of functions for producing a final grinded mixture of ingredients for preparation of an edible composition. In one embodiment, the one or more inputs are received either from a remote server or a mobile application or via a capacitive touch screen display installed on the device. For example, the edible composition may be a batter composition for preparation of Idly, Dosa, Vada and the like.

At step 204, the operation of the plurality of functions is being implemented by the device based on the one or more inputs received at step 202. In one embodiment, the plurality of functions include identifying, weighing, dispensing, cleaning, soaking, grinding, fermenting, altering the proportions and storing of a one or more ingredients for preparation of the edible composition.

In one example, one of the functions of the plurality of functions include grinding the one or more ingredients to produce the final grinded mixture of ingredients for preparation of the edible composition. In one example, one of the functions of the plurality of functions include fermenting the final grinded mixture of ingredients at a controlled temperature. In one example, one of the functions of the plurality of functions include scheduling the operation of each of the plurality of functions of the device.

In another example, one of the functions of the plurality of functions include monitoring a one or more units and its parts of the device for indicating maintenance. In one example, one of the functions of the plurality of functions include monitoring the quality of plurality of sensors and a precise position of the each of the units and its parts of the device. In one example, one of the functions of the plurality of functions include facilitating auto self-cleaning of the each of the units and its parts of the device.

At step 206, a real time information related to the plurality of functions performed by the device, is being communicated to a remote server via a wireless network. In one embodiment, the real time information related to the plurality of functions, is collected and communicated using plurality of sensors, to the remote server via the communication interface.

At step 208, the real time information related to the plurality of functions performed by the device, is analysed by the remote server using artificial intelligence (AI). In one embodiment, Artificial intelligence (AI) is an area of computer science that emphasizes the creation of intelligent machines that work and react like humans. Machine perception deals with the capability to use sensory inputs to deduce the different aspects of the environment. In other embodiment, the remote server is configured for receiving and analyzing the sensed real time information for monitoring a one or more units of the device for indicating maintenance of each of the units of the device.

At step 210, the one or more inputs are generated for the plurality of functions based on analysed information and for making alteration to maintain the taste and quality of the edible composition.

In some embodiments, a manner in which the device 102 of the system 100 of FIG. and its modules/units operates for implementing a plurality of functions based on the one or more inputs received by the remote server 108, is described in detail further below. In some embodiments, the word ‘unit’, ‘module, used in the description may reflect the same meaning and may be used interchangeably.

FIG. 3 illustrates a storage unit 300 of the device 102 of system 100, according to an embodiment of the present disclosure. In particular, the storage unit 300 includes a hopper unit 302 and a controller unit 304 comprising a plurality of sensors 304-A-D.

In one example embodiment, the storage unit 300 includes hopper unit 302 for storing one or more ingredients. For example, rice and pulses are stored in the hopper unit 302 and the hopper container 310 is covered with a hopper lid 308. Over the top of the hopper lid 308, an acrylic lid 306 is placed. The hopper valve 312 is place below the hopper container 310 and it is actuated by a stepper motor to control the gate valve open and close. The hopper container 310 is placed over the hopper base plate 314 which is supported by hopper base lid 316-A-C.

The controller unit 304 consists of a temperature sensor 304-A, a position sensor 304-B, a humidity sensor 304-C and a weight sensor 304-D. The temperature sensor 304-A is used to monitor the temperature in the hopper. The position sensor 304-B is used to monitor the position of hopper valve opening and closing. The humidity sensor 304-C is used to monitor the humidity/water vapor content in the hopper. The weight sensor 304-D is used to monitor the weight of the hopper. In addition, the storage unit 300 includes sensors configured to identify quality of the one or more ingredients. Moreover, the storage unit 300 also includes sensors configured to identify one or more foreign particles.

FIG. 4 illustrates a connecter chamber unit 400 of the device 102 of system 100, according to an embodiment of the present disclosure. In particular, the connector unit 400 includes a counter valve bush 402, stepper motor 404, a hopper base plate 406, a stepper motor mount 408, and a connecting chamber 410.

In one example embodiment, the connecting unit 400 consists of hopper base plate 406 over which the stepper motor mount 408 is placed and on this mount the stepper motor 404 is placed and the counter valve bush 402 is placed in the stepper motor 404. The connecting chamber unit 400 is placed below the hopper base plate which acts as a passage for ingredients (for example, rice/pulses). The connector unit 400 also includes a sensor unit comprising encoders. The encoders are used to translate rotary or linear motion into a digital signal. Usually encoders are used for the purpose of monitoring or controlling motion parameters such as speed, rate, direction, distance or position. The encoder is place over the motor.

FIG. 5 illustrates an electronic module 500 of the device 102 of system 100, according to an embodiment of the present disclosure. In particular, the electronic module 500 includes a battery unit 502, an electronic unit 504 and a processing unit 506.

The battery unit 504 consists of battery 502-A and its wiring 502-B for providing required DC power to the required electronics applications. The display unit consists of display screen which is mounted over the display holder 504-A. The display 504-C is connected to display circuit 504-B to obtain circuit connection. The display shield 504-D is placed before the display screen which acts as a guard for the screen. The processing unit 506 is the heart the machine which has the overall control over the entire circuit and electronic devices. Furthermore, a sensor is connected to the battery unit to analyze the battery life.

FIG. 6 illustrates a motor module 600 of the device 102 of system 100, according to an embodiment of the present disclosure. In particular, the motor module 600 includes a motor unit 602 and a controller unit 604. The motor unit 602 consist of a stepper motor 602-A, grinding motor 602-B, a motor spacer 602-C and a motor bush 602-D. The motor spacer 602-C is placed over the grinding motor 602-B and the grinding bush is placed in the grinding motor shaft. The motor unit 602 also consists of a small stepper motor 602-A to control the opening and closing of the gate place in the grinding container upper lid. The controller unit 604 consists of plurality of sensor like a thermostat 604-A, a speed sensor 604-B and a smoke sensor 604-C. The thermostat 604A is used to monitor the temperature produce by the motor. The speed sensor 604-B is used to measure the speed (RPM) of the motor shaft. The smoke sensor 604-C is used to detect the smoke if any in the device 102.

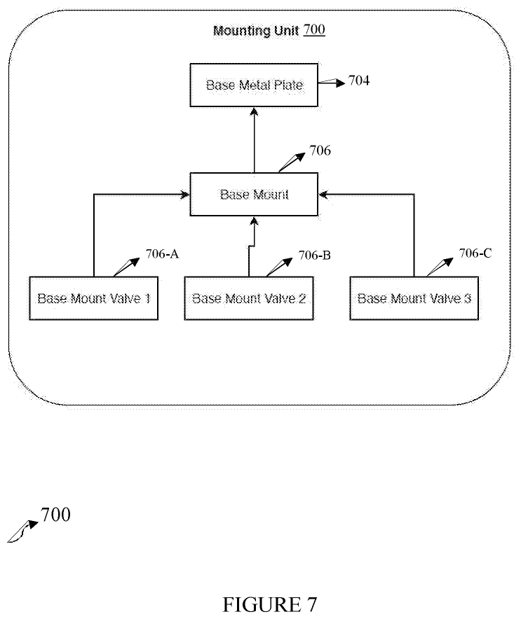

FIG. 7 illustrates a mounting unit 700 of the device 102 of system 100, according to an embodiment of the present disclosure. In particular, the mounting unit 700 includes base mount 706 over which the base mount plate 704 is placed. The base mount 706 is supported by Base mount valve 706-A-C.

FIG. 8 illustrates a grinding module 800 of the device 102 of system 100, according to an embodiment of the present disclosure. In particular, the grinding module 800 includes a grinding unit 802, a controller unit 804 and a grinding blade unit 820. The grinding module 800 consists of a grinding mount on which the grinding container 814 is positioned. The grinding container 814 consists of a lid section which is a combination of upper and lower lid (810 and 812). For example, in this section it has a gate for ingredients (rice/pulses) to fall inside the grinding container 814. The gate door is opened and closed by a gear 808 and piston 806 which is driven by a stepper motor and a bearing is placed below the gear 808 for smooth rotation.

The grinding module 800 consist of blade unit 820 which is an assembly of a blade 820-B and shaft 820-A. The blade unit 820 consists of a blade-grinding connector 820-C and blade stock shaft 820-A, in between which the grinding blade 820-B is placed. The Counter blade holder 820-D which is positioned in the blade-grinding unit 820 and is placed in the blade holder.

The controller unit 804 consists of a position sensor 804-A, a humidity sensor 804-B, a temperature sensor 804-D, a weight sensor 804-C and a PH sensor 804-E. The position sensor 804-A is used to monitor the position of gate door opening and closing. The humidity sensor 804-B is used to monitor the humidity/water vapor content in the grinding container 814. The weight sensor 804-C is used to monitor the weight of the grinding container 814. The temperature sensor 804-D is used to monitor the temperature present inside the grinding container 814. The Ph sensor 804-E is used to monitor the Ph level of water for acidity/base value inside the grinding container 814.

FIG. 9 illustrates a water and fermentation module 900 of the device 102 of system 100, according to an embodiment of the present disclosure. In particular, the water and fermentation module 900 includes a water container unit 902, a controller unit 912 and a fermentation unit 914. The water container lid 906 is placed over the water container 908. The water container 908 also includes a water filter 905 for filtering of water. It has an inlet tube 904 and outlet 910 for water in and out flow. The fermentation and 914 is used to produce hot warm air to ferment the mixture of ingredients (batter) and the refrigeration unit is produce cool air to maintain the refrigeration effect. The controller unit 912 consists of a level sensor 912-A, a temperature sensor 912-B, a Ph sensor 912-C and a microbial sensor configured for monitoring the growth of unwanted microbes and prevent contamination of the water and the final grinded mixture of ingredients.

The temperature sensor 912-B is used to monitor the temperature in the grinding container and water container. The Ph sensor 912-C is used to monitor the ph level of water for acidity/base value.

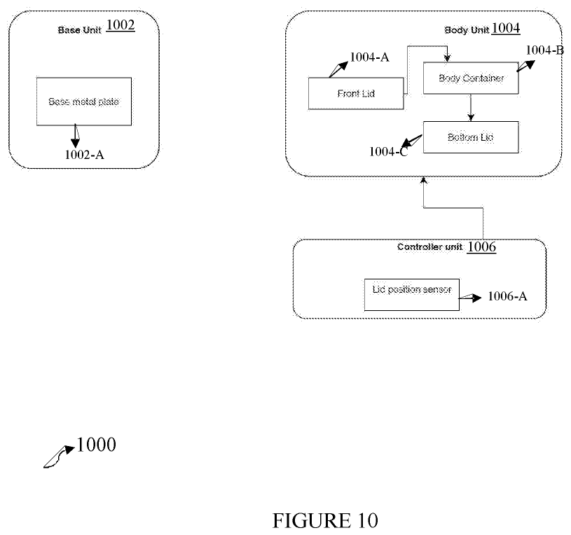

FIG. 10 illustrates a Base and body module 1000 of the device 102 of system 100, according to an embodiment of the present disclosure. The base and body module 1000 consists of base unit 1002, a body unit 1004 and a controller unit 1006. The base unit 1002 is a unit where the base for the entire device 102 is held. The body unit 1004 is the unit where the front body, front body lid 1004-A and entire body of the device is held. The controller unit 1006 consists of lid position sensor 1006-A which is used to sense the position of the lid.

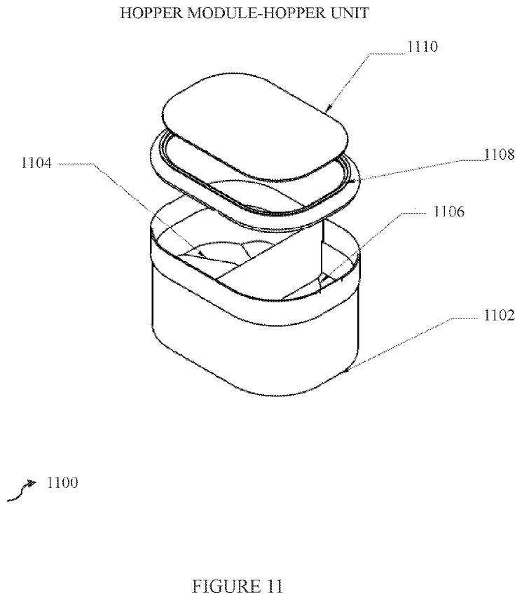

The above described device 102 of the system 100 for performing a plurality of functions like identifying, weighing, dispensing, cleaning, soaking, grinding, fermenting, altering the proportions and storing of a one or more ingredients for preparation of an edible composition, based on a one or more inputs received from a remote server 108 includes a plurality of units. Some example cross-sectional views of the plurality of units and the overall assembly of the device is described and illustrated in FIGS. 11 through 21. It is noted that the present technique is by no means intended to be limited to these example embodiments.

FIGS. 11 through 21 show cross-sectional views illustrating several ‘units/modules’ of the device 102 of system 100 of FIG. 1, implemented according an embodiment of the present disclosure.

FIG. 11 illustrates a cross-sectional view of a hopper unit 1100 of a hopper module of the device 102 of system 100 of FIG. 1, implemented according an embodiment of the present disclosure. In particular, FIG. 11 illustrates a hopper 1102, a Rice 1104, urud dhal 1106, a lid 1108 and an acrylic lid 1110.

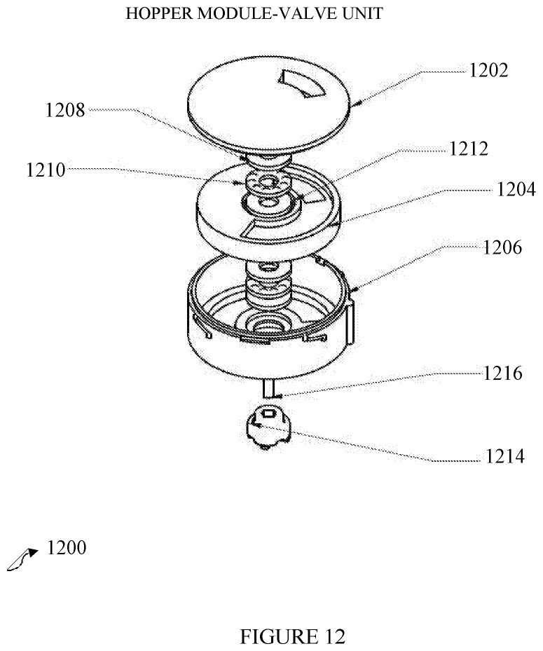

FIG. 12 illustrates a cross-sectional view of a valve unit 1200 of a hopper module of the device 102 of system 100 of FIG. 1, implemented according an embodiment of the present disclosure. In particular, FIG. 12 illustrates a lid 1202, a valve 1204, a shell 1206, a thrush bearing 1208, a ball bearing 1210, a thrush bearing 1212, a bush 1214 and a bush connector 1216.

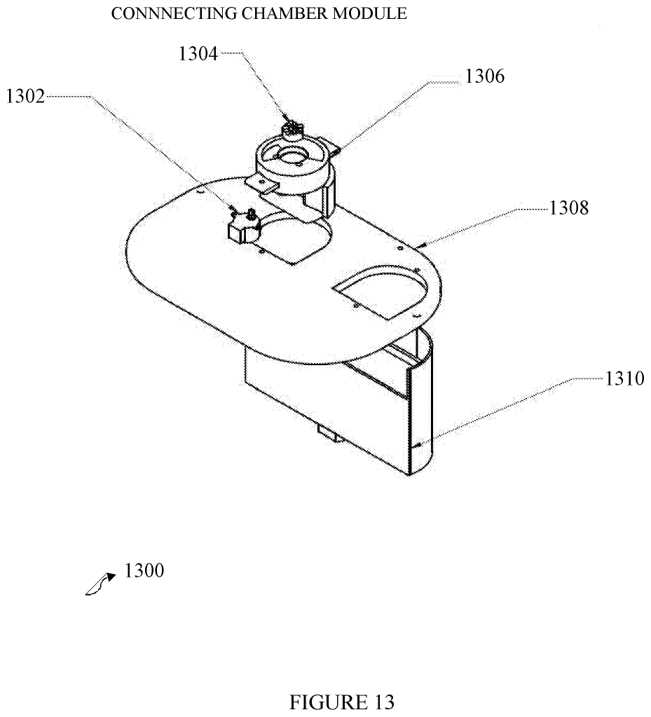

FIG. 13 illustrates a cross-sectional view of a connecting chamber module 1300 of the device 102 of system 100 of FIG. 1, implemented according an embodiment of the present disclosure. In particular, FIG. 13 illustrates a stepper motor 1302, a counter valve bush 1304, a storage motor mount 1306, a hopper base plate 1308 and a connecting chamber 1310.

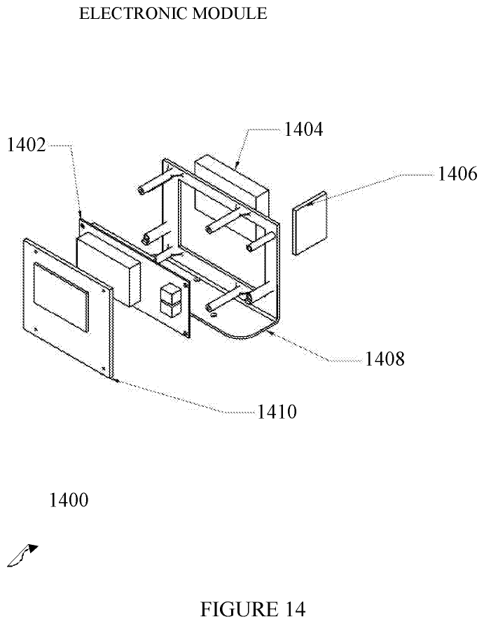

FIG. 14 illustrates a cross-sectional view of an electronic module of the device 102 of system 100 of FIG. 1, implemented according an embodiment of the present disclosure. In particular, FIG. 14 illustrates a display 1402, a sensor unit 1404, a Wifi module 1406, a display holder 1408 and a display shield 1410.

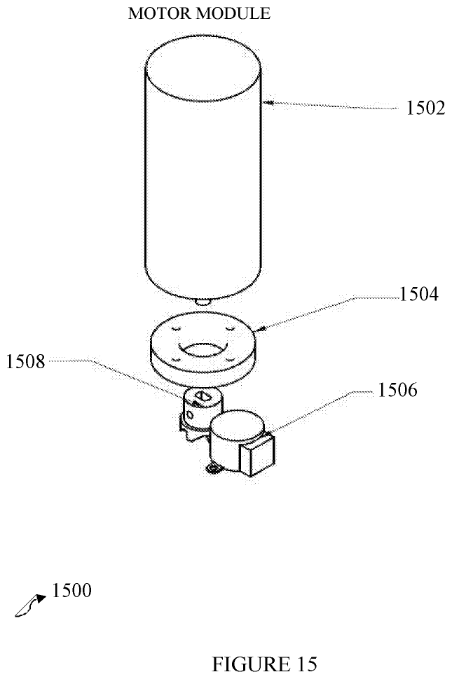

FIG. 15 illustrates a cross-sectional view of a motor module of the device 102 of system 100 of FIG. 1, implemented according an embodiment of the present disclosure. In particular, FIG. 15 illustrates a grinding motor 1502, a motor spacer 1504, a stepper motor 1506 and a grinding bush 1508.

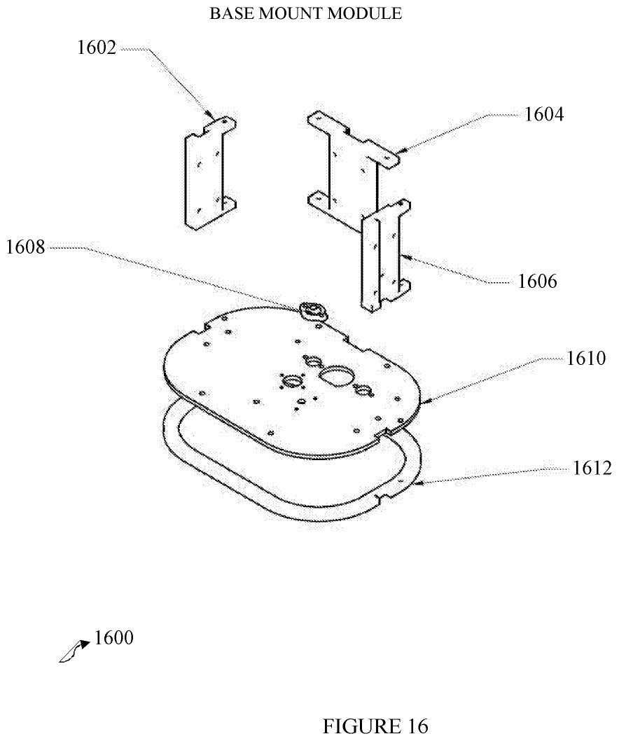

FIG. 16 illustrates a cross-sectional view of a base mount module of the device 102 of system 100 of FIG. 1, implemented according an embodiment of the present disclosure. In particular, FIG. 16 illustrates a hopper base lid 3, 1602, a hopper base lid 2, 1604, a hopper base lid 1606, a water tube mount 1608, a base mount plate 1610 and a base mount 1612.

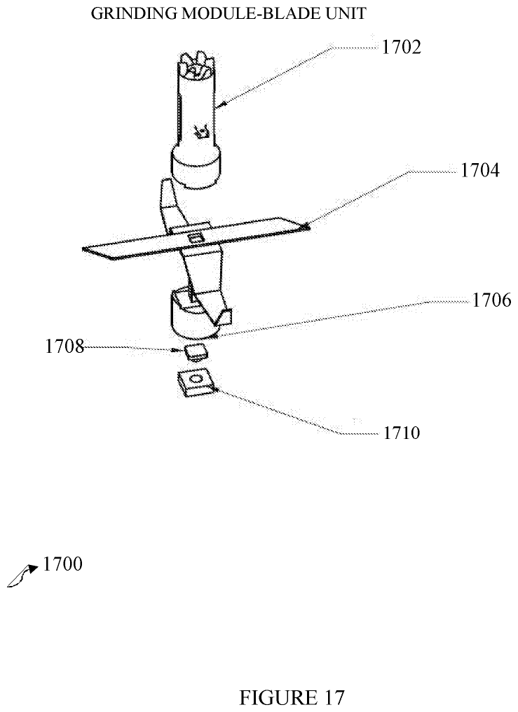

FIG. 17 illustrates a cross-sectional view of a blade unit of a grinding module of the device 102 of system 100 of FIG. 1, implemented according an embodiment of the present disclosure. In particular, FIG. 17 illustrates a blade stock shaft 1702, a blade 1704, a blade-grinding unit connector 1706, a counter blade holder 1708 and a blade holder 1710.

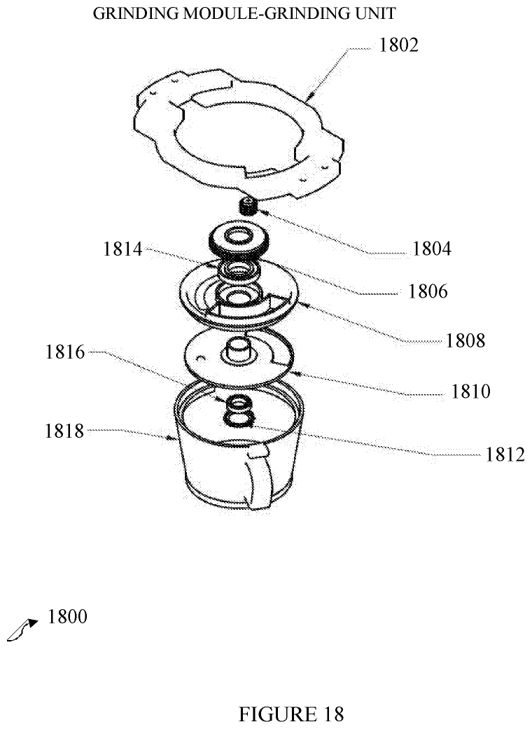

FIG. 18 illustrates a cross-sectional view of a grinding unit of a grinding module of the device 102 of system 100 of FIG. 1, implemented according an embodiment of the present disclosure. In particular, FIG. 18 illustrates a grinding mount 1802, a pinion 1804, a gear 1806, an upper lid 1808, a lower lid 1810, a bearing cap 1812, a bearing 1814, a lower bearing 1816 and a grinding container 1818.

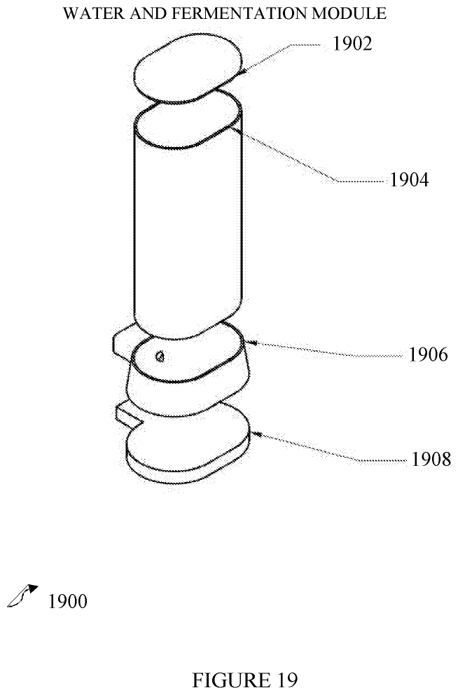

FIG. 19 illustrates a cross-sectional view of a water and a fermentation module of the device 102 of system 100 of FIG. 1, implemented according an embodiment of the present disclosure. In particular, FIG. 19 illustrates a water container lid 1902, a water container 1904, a water container holder 1906 and a fermentation and a refrigeration unit 1908.

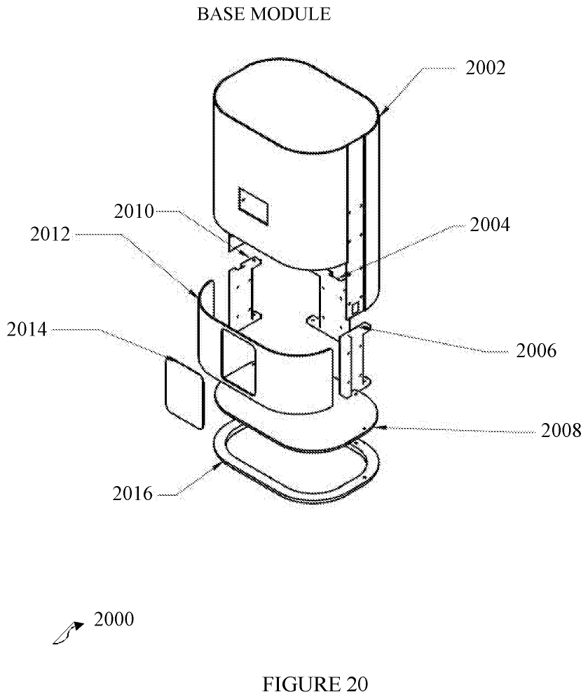

FIG. 20 illustrates a cross-sectional view of a base module of the device 102 of system 100 of FIG. 1, implemented according an embodiment of the present disclosure. In particular, FIG. 20 illustrates a body 2002, a base mount valve 2, 2004, a base mount valve 1, 2006, a base metal plate 2008, a base mount valve 3, 2010, a front body 2012, a front body lid 2014 and a base 2016.

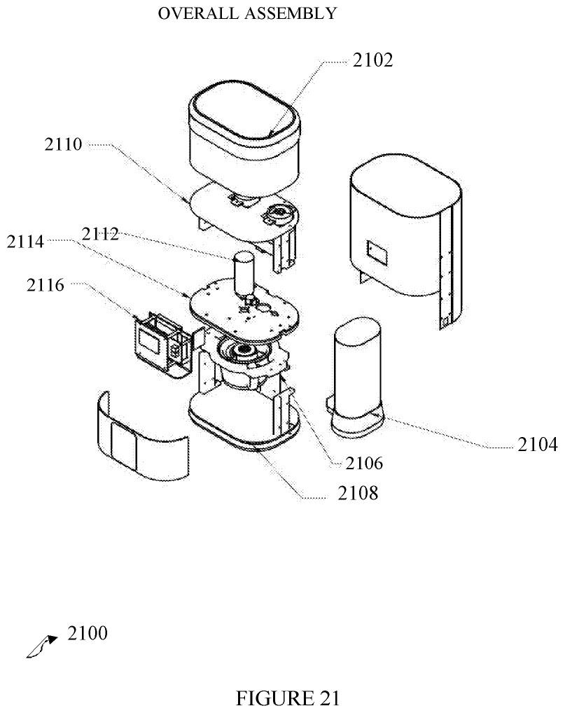

FIG. 21 illustrates a cross-sectional view of an overall assembly 2100 of the device 102 of system 100 of FIG. 1, implemented according an embodiment of the present disclosure. In particular, FIG. 21 illustrates a storage module 2102, a water and a fermentation module 2104, a grinding unit module 2106, a base module 2108, a connecting chamber module 2110, a motor module 2112, a base mount module 2114 and an electronic module 2116.

While specific language has been used to describe the disclosure, any limitations arising on account of the same are not intended. As would be apparent to a person skilled in the art, various working modifications may be made to the method in order to implement the inventive concept as taught herein. The figures and the foregoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, orders of processes described herein may be changed and are not limited to the manner described herein. Moreover, the actions of any flow diagram need not be implemented in the order shown; nor do all of the acts necessarily need to be performed. Also, those acts that are not dependent on other acts may be performed in parallel with the other acts. The scope of embodiments is by no means limited by these specific examples. Numerous variations, whether explicitly given in the specification or not, such as differences in structure, dimension, and use of material, are possible. The scope of embodiments is at least as broad as given by the following claims.

I/We claim:

1. A system comprising:
a device configured for performing a plurality of functions based on a one or more inputs received; wherein the plurality of functions comprises identifying, weighing, dispensing, cleaning, soaking, grinding, fermenting, altering the proportions and storing of a one or more ingredients for preparation of an edible composition;
a communication interface configured for communicating the one or more inputs to the device and further communicating a real time information related to the plurality of functions performed, to a remote server via a wireless network; and
the remote server configured for continuously analysing the real time information related to the plurality of functions performed by the device by using artificial intelligence (AI) for generating the one or more inputs for the plurality of functions.

2. The system of claim 1, wherein the device comprises a plurality of units; wherein the plurality of units further comprises a plurality of sensors configured for collecting and communicating real time information related to the plurality of functions to the remote server via the communication interface for the artificial intelligence (AI) to analyse and communicate inputs.

3. The system of claim 2, wherein the plurality of units are:
a storage unit; wherein the storage unit comprises:
a one or more hoppers configured for storing the one or more ingredients separately and free from contamination; and
the plurality of sensors configured for: identifying the quality of one or more ingredients, weighing the one or more ingredients stored in the hopper, monitoring the temperature in the hopper, checking a position of a hopper valve opening and closing, monitoring humidity and water vapour content in the hopper and further communicating the real time sensed information to the remote server via the communication interface for the artificial intelligence (AI) to perform alteration for maintaining the taste and quality of the edible composition;
a connecting unit configured for a passage of the one or more ingredients to a grinding unit; wherein the connecting unit comprises encoders configured to translate rotary or linear motion into a digital signal;
an electronic unit comprising: a battery for sustaining the operation of the device during power failure; a power regulation unit for monitoring any fluctuation in power using current and voltage sensors; a display and a processing unit; and
a motor unit comprising:
a grinding motor configured for grinding the one or more ingredients to produce a final grinded mixture of ingredients for preparation of an edible composition and
the plurality of sensors configured for:
monitoring the temperature produced by the grinding motor
measuring the speed (RPM) of a motor shaft of the grinding motor; and
detecting a smoke.

4. The system of claim 2, wherein the device further comprises:
a soaking unit configured for soaking the one or more ingredients using fresh water from a water container comprising water level sensors;
a fermentation unit configured to produce hot warm air for fermenting the final grinded mixture of the ingredients; and
a refrigeration unit configured to produce cool air for maintaining a refrigeration effect for the final grinded mixture of the ingredients.

5. The system of claim 2, wherein the device further comprises a controller unit comprising:
a temperature sensor configured for monitoring the temperature in a grinding container and a water container;
a PH sensor configured for monitoring the Ph level of the water for acid and base value and for further monitoring the acid and base value of the final grinded mixture of ingredients;
a microbial sensor configured for monitoring the growth of unwanted microbes and prevent contamination of the water and the final grinded mixture of ingredients; and
a gas sensor, a pressure sensor and a taste bud sensor configured for collecting physiochemical parameters of the final grinded mixture of ingredients.

6. The system of claim 2, wherein the device further comprises:
a sensor management module of the circuit for monitoring the quality of plurality of sensors; and
a plurality of lock sensors configured for monitoring the precise position of the several units and its parts.

7. The system of claim 2, wherein the device facilitates auto self-cleaning of the plurality of units and its parts.

8. The system of claim 1, wherein the one or more inputs are received either from a remote server or a mobile application or via a capacitive touch screen display installed on the device.

9. The system of claim 1, wherein the remote server is configured for scheduling the operation of each of the functions of the device.

10. The system of claim 1, wherein the remote server is configured for monitoring a one or more units of the device for indicating maintenance of each of the units of the device.

11. The system of claim 1, wherein the device is a smart grinding and blending device communicatively coupled to the remote server further enabling a user to send one or more inputs from a hand-held device via an interface.

12. The system of claim 11, wherein the device is an IoT (internet of things) enabled device configured for remote operation using handheld devices.

13. A method comprising:
communicating the one or more inputs to a device, for performing a plurality of functions for producing a final grinded mixture of ingredients for preparation of an edible composition;
implementing the operation of the plurality of functions, by the device; wherein the plurality of functions comprises identifying, weighing, dispensing, cleaning, soaking, grinding, altering the proportions, fermenting and storing of a one or more ingredients for preparation of an edible composition;
communicating a real time information related to the plurality of functions performed, to a remote server via a wireless network; and
analysing the real time information by the remote server, related to the plurality of functions performed by the device by using artificial intelligence (AI) for generating the one or more inputs for the plurality of functions and for making alteration to maintain the taste and quality of the edible composition.

14. The method of claim 13, further comprising grinding the one or more ingredients to produce a final grinded mixture of ingredients for preparation of an edible composition.

15. The method of claim 13, further comprising fermenting the final grinded mixture of ingredients at a controlled temperature.

16. The method of claim 13, further comprising scheduling the operation of each of the plurality of functions of the device.

17. The method of claim 13, further comprising monitoring a one or more units and its parts of the device for indicating maintenance.

18. The method of claim 13, further comprising collecting and communicating real time information using plurality of sensors, related to the plurality of functions to the remote server via the communication interface for the artificial intelligence (AI) to analyse and communicate inputs.

19. The method of claim 13, further comprising monitoring the quality of plurality of sensors and a precise position of the each of the units and its parts of the device.

20. The method of claim 13, further comprising facilitating auto self-cleaning of the each of the units and its parts of the device.
